(19) United States
(12) Patent Application Publication
(10) Pub. No.: US 2010/0222151 A1
(43) Pub. Date: Sep. 2, 2010

(54) APPARATUS FOR MAINTAINING AN ANGULAR POSITION OF A LEG

(76) Inventor: Thomas MCNAMARA, North Tonawanda, NY (US)

Correspondence Address: SIMPSON & SIMPSON, PLLC, 5555 MAIN STREET, WILLIAMSVILLE, NY, 14221-5406, US

(21) Appl. No.: 12/394,355

(22) Filed: Feb. 27, 2009

(51) Int. Cl. A63B 69/36; A63B 69/00; A61F 5/00

(52) U.S. Cl. 473/207; 473/458; 602/26; 473/409

(57) ABSTRACT

An apparatus for maintaining an angular position of a leg having a knee, the apparatus including a rigid frame having oppositely disposed first and second side walls and an integral connecting member therebetween, each of the first and second side walls includes a plurality of through holes, a substantially rigid support member releasably secured to one of the plurality of through holes of the first side wall and one of the plurality of through holes of the second side wall, the support member positioned behind knee, and first and second securing straps adapted to be releasably secured about the leg above and below the knee, respectively, wherein modifying a position of the substantially rigid support member relative to the first and second side walls modifies the angular position of the leg.

### FIELD OF THE INVENTION

The invention broadly relates to support apparatus, more specifically to a support apparatus for a knee, and even more particularly to an apparatus for maintaining an angular position of a leg.

### BACKGROUND OF THE INVENTION

Sport activities which require swinging an object with the upper body while maintaining the lower body in a relatively fixed location demand both a high level of skill and consistent positioning of the lower body. Examples of such activities include but are not limited to golf, baseball, cricket and the like. While participating in these activities, the lower body must be maintained in what is commonly referred to as an athletic stance, i.e., feet positioned approximately shoulder width apart, knees flexed and a slight bend forward at the hips/waist. Although activities such as baseball and cricket also require a player to run after striking a ball, the lower body positioning is quite similar to other activities which do not require subsequent running, e.g., golf.

It is common for players to have difficulty maintaining proper position and bend to the knees, and in particular the back knee, i.e., the knee farthest away from the direction of striking the ball. Improper straightening of the rear or back knee can cause a player to miss the ball or to hit the ball in an undesirable direction. Thus, many devices and apparatus have been developed to assist with maintaining the proper bend of a player's knee during both practice and actual play.

For example, U.S. Pat. No. 5,800,371 (Winn) discloses a sportman's knee brace that is applied to the back of the knee for inducing a player to assume a correct swinging stance. The Winn brace comprises a rigid, padded, oblong plate folded outwardly to an obtuse angle and is secured to the thigh, lower knee and calf of the user by a series of straps. Depending on the particular user's needs, the angle of the device may be modified by bending the oblong plate to a different angle. Thus, each time the device is modified to a new position, the plate material is deteriorated, as one of ordinary skill in the art appreciates that flexing, especially repeated flexing, of a material tends to breakdown the material to the extent that the material will eventually fail.

A further example is of this type of device is described in U.S. Pat. No. 6,672,973 (Muntz). Muntz discloses a golf training device comprising a first strap fastened around a golfer's leg above the knee, a second strap fastened above the ankle and below the calf muscle, and a third strap joined to and interconnecting both the first and second straps. The third strap runs vertically up behind the leg to a type of a quick-release adjusting clasp, and its length can be shortened by pulling on an adjusting strap, thereby causing the leg to bend at the knee. A tube is positioned behind the golfer's knee to ensure that there is also a horizontal force on the first strap which helps to securely hold the first strap in place and to prevent it from slipping down the leg. The device causes the knee to remain flexed throughout the full swing of a golf club; however, the device fails to prevent the knee from bending too far as the device merely comprises flexible strap material and not a rigid frame structure.

Similarly, U.S. Pat. Nos. 6,361,448; 6,692,369; and, 7,163,464 (McKeon) each disclose an apparatus and method for controlling and stabilizing the swing mechanics of a golfer. The apparatus and method provide means to maintain the necessary critical knee flexion angle throughout the golf swing, to properly transfer the weight of the golfer and to properly load the necessary side of the golfer for a reliable, consistent, controlled and safe golf swing. The apparatus comprises upper and lower leg support members and an adjustable hinge about the knee flexion axis that accommodates variable degrees of flexion depending on golfer specific conditions, e.g., height, arm and leg length, weight, etc., and environmental conditions, e.g., course conditions, ball lie, playing hazards, etc.. The adjustable hinge includes both a locked position mode that prevents the knee from flexing and an unlocked position mode that permits unrestricted movement. The apparatus taught in these patents includes a complex and expensive means of maintaining the angular position of a leg, i.e., the adjustable hinge mechanism.

As can be derived from the variety of devices and methods directed at maintaining an angular position of a leg, many means have been contemplated to accomplish the desired end, i.e., consistent and convenient control of the angular position of a leg. Heretofore, tradeoffs between device complexity/expense and cost were required. Thus, there is a long-felt need for an apparatus for maintaining the angular position of a leg that is simple to construct, easy to use and cost effective to produce. There is a further long-felt need for a simple and consistent method of maintaining the angular position of a leg throughout a swinging motion.

### BRIEF SUMMARY OF THE INVENTION

The present invention broadly comprises an apparatus for maintaining an angular position of a leg having a knee, the apparatus including a rigid frame having oppositely disposed first and second side walls and an integral connecting member therebetween. The integral connecting member is arranged to connect at least a portion of the first side wall to at least a portion of the second side wall and to be positioned behind the knee. Each of the first and second side walls includes a plurality of through holes wherein the plurality of through holes of the first sidewall are in registered alignment with the plurality of through holes of the second side wall. The apparatus further includes a substantially rigid support member releasably secured to one of the plurality of through holes of the first side wall and one of the plurality of through holes of the second side wall and the support member is positioned behind the knee. Furthermore, the apparatus includes first and second securing straps. The first securing strap is releasably secured to upper portions of the first and second side walls, while the second securing strap is releasably secured to lower portions of the first and second side walls. The first strap is adapted to be releasably secured about a portion of the leg above the knee, while the second strap is adapted to be releasably secured about a portion of the leg below the knee. In the foregoing embodiment, the angular position of the leg is modified by altering the position of the substantially rigid support member relative to the first and second side walls.

In some embodiments, the first and second side walls and the connecting member form a substantially U-shaped cross section. In other embodiments, a shape of the substantially rigid support member is selected from the group consisting of: a cylinder, a hemi-cylinder, a crescent cylinder, a sphere, a hemisphere, a cuboid and combinations thereof. In still yet other embodiments, each of the first and second side walls are arranged on opposite sides of the leg above and below the knee. In yet further embodiments, each of the plurality of through holes in the first side wall is arranged at a unique horizontal and/or vertical position relative to the others of the plurality of through holes in the first side wall and each of the plurality of through holes in the second side wall is arranged at a unique horizontal and/or vertical position relative to the others of the plurality of through holes in the second side wall. In some of these embodiments, the unique positions of the plurality of through holes permits modifying the position of the substantially rigid support member.

In some embodiments, the substantially rigid support member is releasably secured to the first and second side walls via a securing means and the securing means is selected from the group consisting of: a longitudinal shaft disposed through the support member, an integral threaded shaft disposed at each end of the support member, a spring loaded ball detent disposed at each end of the support member, a locking pin disposed in each end of the support member and combinations thereof. In further embodiments of the present invention apparatus, the substantially rigid support member includes a single material, while in other embodiments, the substantially rigid support member includes a core enclosed within an outer shell. In still yet other embodiments, each of the first and second securing straps includes hook and loop type fasteners, and in still further embodiments, each of the first and second securing straps includes a plurality of holes disposed along at least a portion of the strap and a retaining means disposed at a terminal end of the strap.

The present invention also broadly comprises a method of maintaining an angular position of a leg having a knee. The method includes the steps of: a) providing a rigid frame having oppositely disposed first and second side walls and an integral connecting member therebetween, the integral connecting member arranged to connect at least a portion of the first side wall to at least a portion of the second side wall and to be positioned behind the knee; b) providing a plurality of through holes in each of the first and second side walls, the plurality of through holes of the first sidewall in registered alignment with the plurality of through holes of the second side wall; c) providing a substantially rigid support member releasably secured to one of the plurality of through holes of the first side wall and one of the plurality of through holes of the second side wall, the support member positioned behind the knee; d) providing first and second securing straps, the first securing strap is releasably securable to upper portions of the first and second side walls and the second securing strap is releasably securable to lower portions of the first and second side walls, the first strap is adapted to be releasably secured about a portion of the leg above the knee and the second strap is adapted to be releasably secured about a portion of the leg below the knee; e) positioning the connecting member and the substantially rigid support member behind the knee; and, f) affixing the first and second securing straps about the leg, wherein modifying a position of the substantially rigid support member relative to the first and second side walls modifies the angular position of the leg.

It is a general object of the present invention to provide an apparatus for maintaining the angular position of a leg that provides consistent and convenient control of the angular position of the leg.

It is another general object of the present invention to provide an apparatus for maintaining the angular position of a leg that is simple to use, and inexpensive and easy to manufacture.

It is yet another object of the present invention to provide a method of maintaining the angular position of a leg throughout a swinging motion that is both effective and convenient to perform.

These and other objects and advantages of the present invention will be readily appreciable from the following description of preferred embodiments of the invention and from the accompanying drawings and claims.

### BRIEF DESCRIPTION OF THE DRAWINGS

The nature and mode of operation of the present invention will now be more fully described in the following detailed description of the invention taken with the accompanying drawing figures, in which:

FIG. 1 is a side elevational view of an embodiment of a present invention apparatus for maintaining an angular position of a leg secured to a leg of a golfer;

FIG. 2 is an enlarged side elevational view of an embodiment of a present invention apparatus for maintaining an angular position of a leg secured to a leg of a golfer having a support member in a first position;

FIG. 3 is an enlarged side elevational view of an embodiment of a present invention apparatus for maintaining an angular position of a leg secured to a leg of a golfer having a support member in a second position;

FIG. 4 is a back elevational view of an embodiment of a present invention apparatus for maintaining an angular position of a leg;

FIG. 4A is a cross sectional view of the apparatus of FIG. 4 taken generally along Line 4A-4A of FIG. 4;

FIG. 4B is a cross sectional view of the apparatus of FIG. 4 taken generally along Line 4B-4B of FIG. 4 with the support member removed for purposes of discussion;

FIG. 5 is a front elevational view of an embodiment of a present invention apparatus for maintaining an angular position of a leg;

FIG. 6 is a top plan view of an embodiment of a present invention apparatus for maintaining an angular position of a leg;

FIG. 7 is an exploded front elevational view of an embodiment of a present invention apparatus for maintaining an angular position of a leg;

FIG. 8A is a side elevational view of an embodiment of a support member for use in a present invention apparatus for maintaining an angular position of a leg;

FIG. 8B is a side elevational view of another embodiment of a support member for use in a present invention apparatus for maintaining an angular position of a leg;

FIG. 8C is a side elevational view of yet another embodiment of a support member for use in a present invention apparatus for maintaining an angular position of a leg;

FIG. 8D is a side elevational view of still yet another embodiment of a support member for use in a present invention apparatus for maintaining an angular position of a leg;

FIG. 8E is a side elevational view of a further embodiment of a support member for use in a present invention apparatus for maintaining an angular position of a leg;

FIG. 9A is a cross sectional view of an embodiment of a support member for use in a present invention apparatus for maintaining an angular position of a leg;

FIG. 9B is a cross sectional view of another embodiment of a support member for use in a present invention apparatus for maintaining an angular position of a leg;

FIG. 10A is a cross sectional view of the apparatus of FIG. 5 taken generally along Line 10A/B-10A/B of FIG. 5 having an embodiment of a means for securing a support member within the rigid frame of the present invention apparatus; and,

FIG. 10B is a cross sectional view of the apparatus of FIG. 5 taken generally along Line 10A/B-10A/B of FIG. 5 having another embodiment of a means for securing a support member within the rigid frame of the present invention apparatus.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

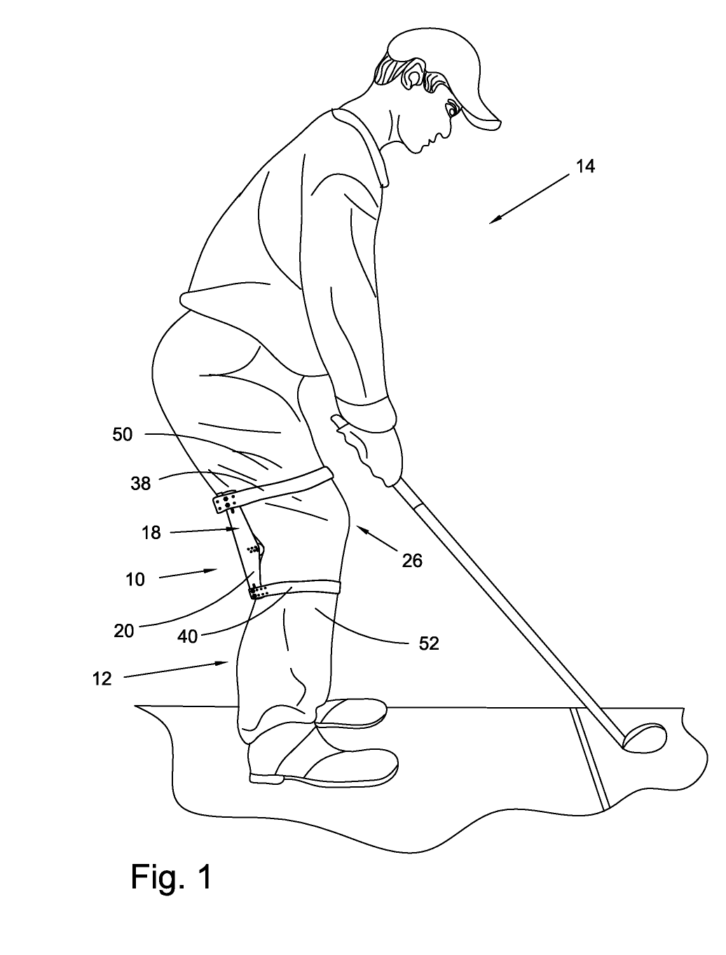

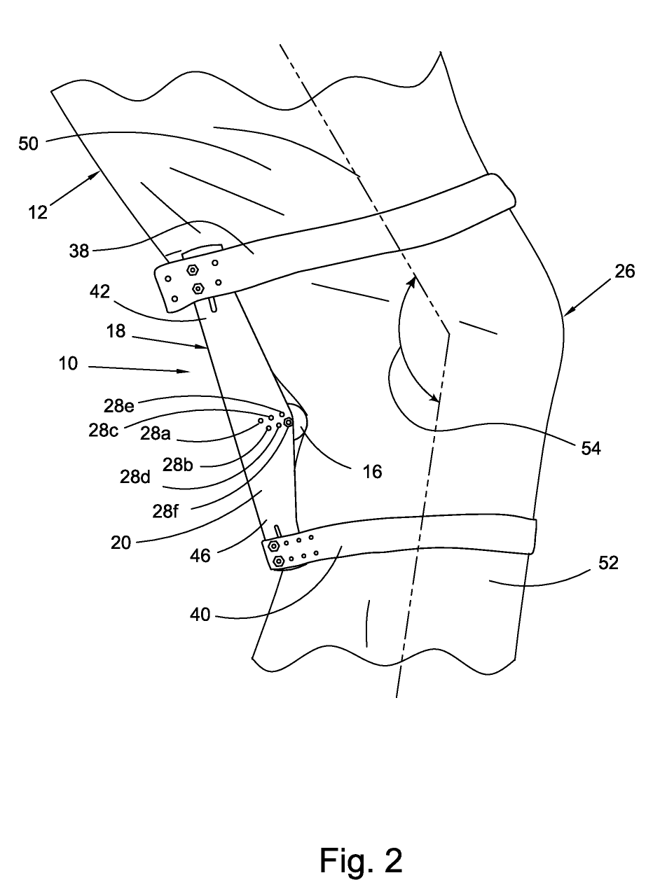

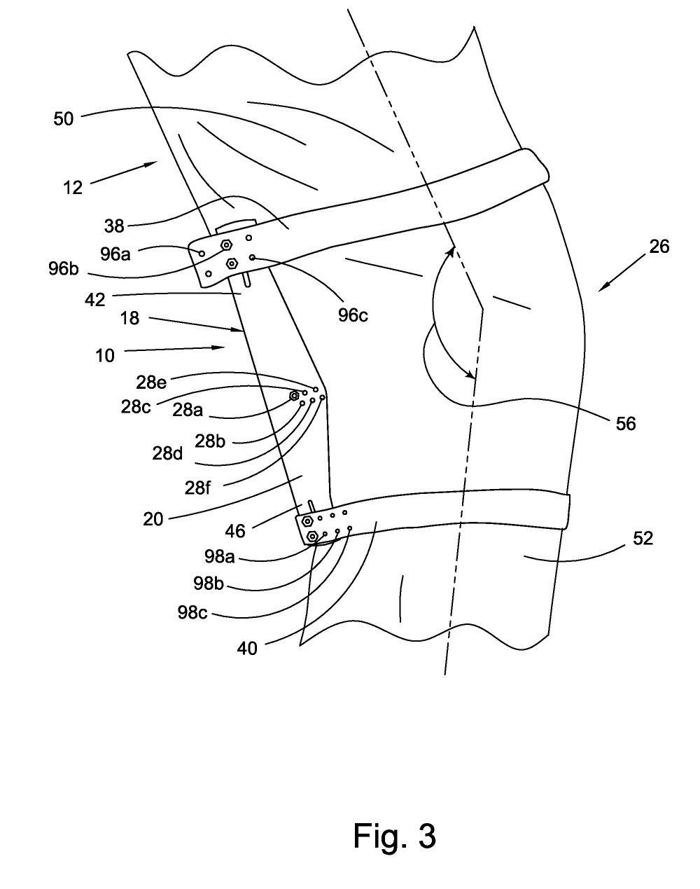

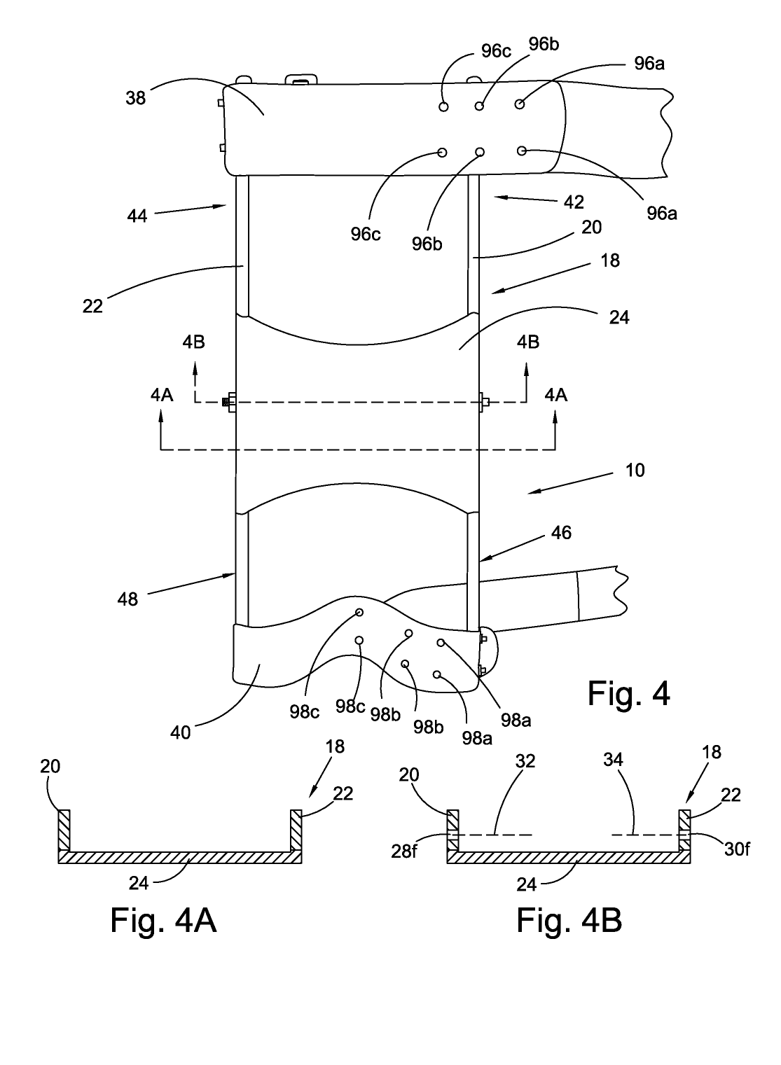

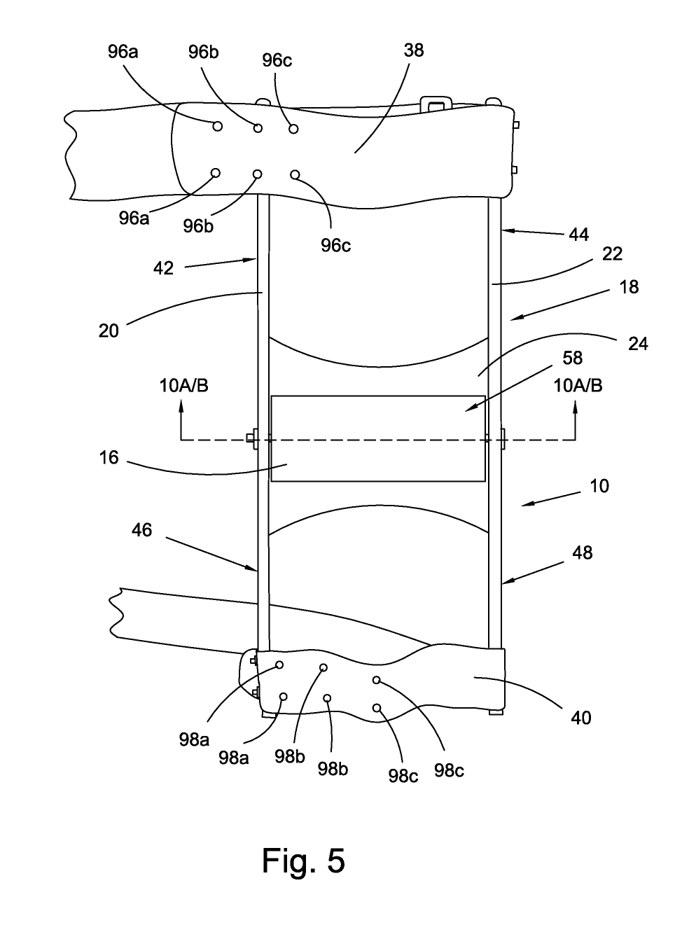

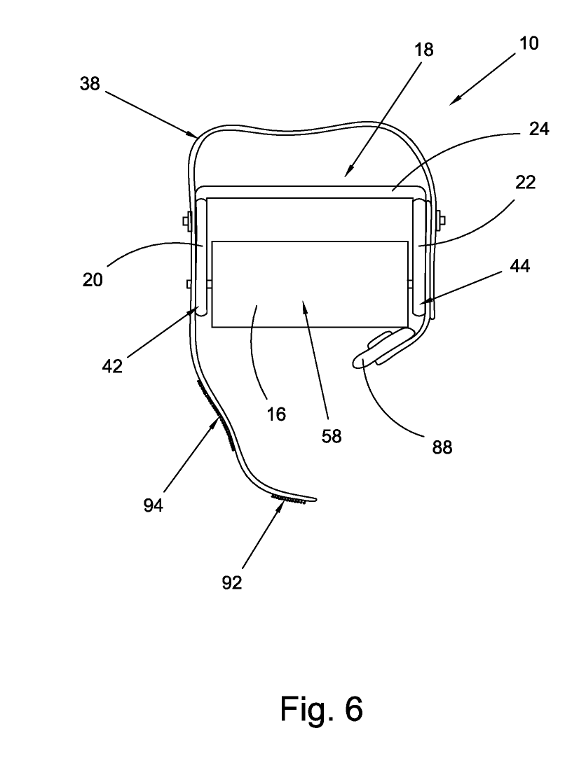

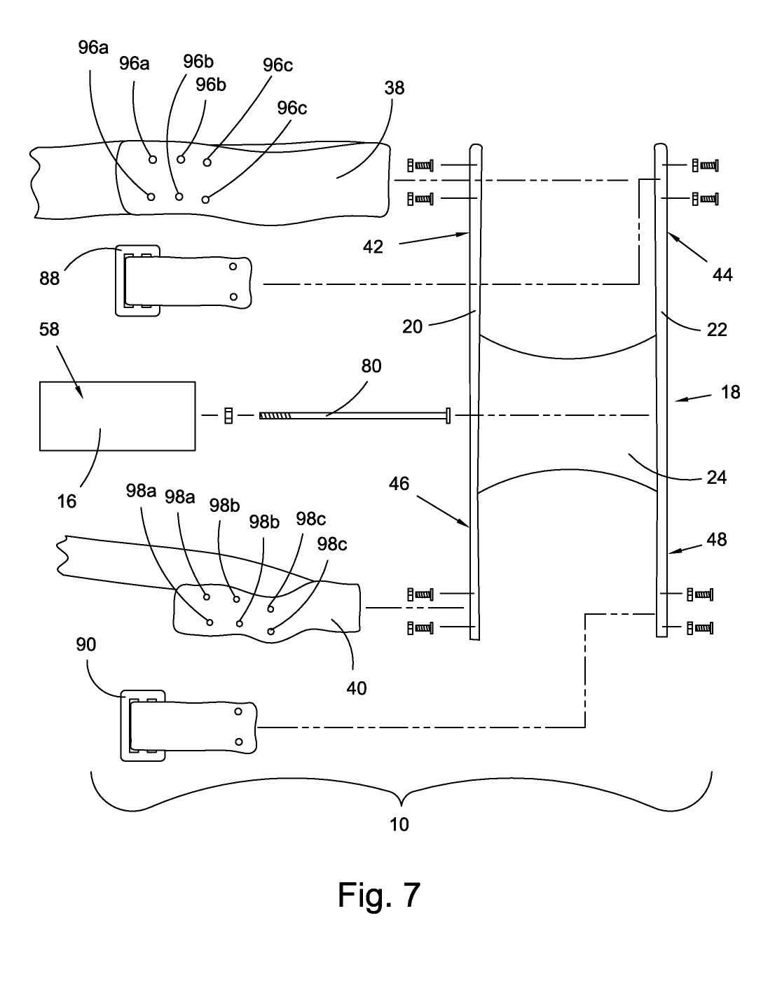

Adverting now to the figures, FIG. 1 shows a side elevational view of an embodiment of a present invention apparatus for maintaining an angular position of a leg, i.e., apparatus 10, secured to leg 12 of golfer 14. FIG. 2 shows an enlarged side elevational view of apparatus 10 secured to leg 12 having support member 16 in a first position, while FIG. 3 shows an enlarged side elevational view of apparatus 10 secured to leg 12 having support member 16 in a second position. FIG. 4 shows a back elevational view of apparatus 10, while FIG. 4A shows a cross sectional view of apparatus 10 taken generally along Line 4A-4A of FIG. 4 and FIG. 4B shows a cross sectional view of apparatus 10 taken generally along Line 4B-4B of FIG. 4 with support member 16 removed for purposes of discussion. FIG. 5 shows a front elevational view of apparatus 10, while FIG. 6 shows a top plan view of apparatus 10. Lastly, FIG. 7 shows an exploded front elevational view of apparatus 10. The following discussion is best understood in view of FIGS. 1 through 7.

Apparatus 10 comprises rigid frame 18. Rigid frame 18 comprises oppositely disposed first and second side walls 20 and 22, respectively, and integral connecting member 24 therebetween. Integral connecting member 24 is arranged to connect at least a portion of first side wall 20 to at least a portion of second side wall 22 and to be positioned behind knee 26. It should be appreciated that although in the figures, connecting member 24 is shown to connect only a portion of first and second side walls 20 and 22, respectively, connecting member 24 may connect the entire length of first and second side walls 20 and 22, respectively, and such variations are within the spirit and scope of the claimed invention. As is further appreciated in view of the figures, each of first and second side walls 20 and 22, respectively, is arranged on opposite sides of leg 12 above and below knee 26. Each of first and second side walls 20 and 22, respectively, comprises a plurality of through holes, e.g., through holes 28a, 28b, 28c, 28d, 28e, 28f, and through holes 30a, 30b, 30c, 30d, 30e and 30f, respectively. Except when discussed individually, hereinafter through holes 28a through 28f are collectively referred to as through holes 28, while through holes 30a through 30f are collectively referred to as through holes 30. Through holes 28 of first sidewall 20 are in registered alignment with through holes 30 of second side wall 22. The registered alignment of through holes 28 and 30 is best understood in view of FIG. 4B. As can be seen in that figure, central axis 32 of through hole 28f is coaxial with central axis 34 of through hole 30f. Thus, it should be appreciated that each through hole in first side wall 20 has an aligned counter through hole in second side wall 22, and that although all of through holes 30 are not shown, second side wall 22 contains an identical pattern of through holes as first side wall 20. Apparatus 10 further comprises substantially rigid support member 16 which is releasably secured to one of through holes 28 of first side wall 20 and one of through holes 30 of second side wall 22. Support member 16 is positioned behind knee 26. Moreover, apparatus 10 still yet further comprises first and second securing straps 38 and 40, respectively. First securing strap 38 is releasably secured to upper portion 42 of first side wall 20 and upper portion 44 of second side wall 22. Second securing strap 40 is releasably secured to lower portion 46 of first side wall 20 and lower portion 48 of second side wall 22. First strap 38 is adapted to be releasably secured about portion 50 of leg 12 above knee 26, while second strap 40 is adapted to be releasably secured about portion 52 of leg 12 below knee 26.

In the foregoing embodiment, modifying the position of support member 16 relative to first and second side walls 20 and 22, respectively, modifies the angular position of leg 12. This aspect of the invention is best understood in view FIGS. 2 and 3. In FIG. 2, support member 16 is located and disposed between through holes 28f and 30f. With such an arrangement, knee 26 is held at a first angular position, i.e., angle 54. Contrarily, in FIG. 3, support member 16 is located and disposed between through holes 28a and 30a. With such an arrangement, knee 26 is held at a second angular position, i.e., angle 56. Due to the foregoing arrangements, knee 26 is bent more in FIG. 2 than in FIG. 3, or in other words, angle 54 is less than angle 56. It should be appreciated that in some embodiments, the angular position of leg 12 and therefore knee 26 are controlled by the placement of support member 16 relative to first and second side walls 20 and 22, respectively, or in other words, relative to through holes 28 and 30. Thus, each of through holes 28 in first side wall 20 is arranged at a unique horizontal and/or vertical position relative to the others of through holes 28, and each of through holes 30 in second side wall 22 is arranged at a unique horizontal and/or vertical position relative to the others of through holes 30. It is the unique positions of through holes 28 and 30 which permit modifying the position of substantially rigid support member 16. Additionally, as can be seen in FIG. 4A, in some embodiments of the invention, first and second side walls 20 and 22, respectively, and connecting member 24 form a substantially U-shaped cross section.

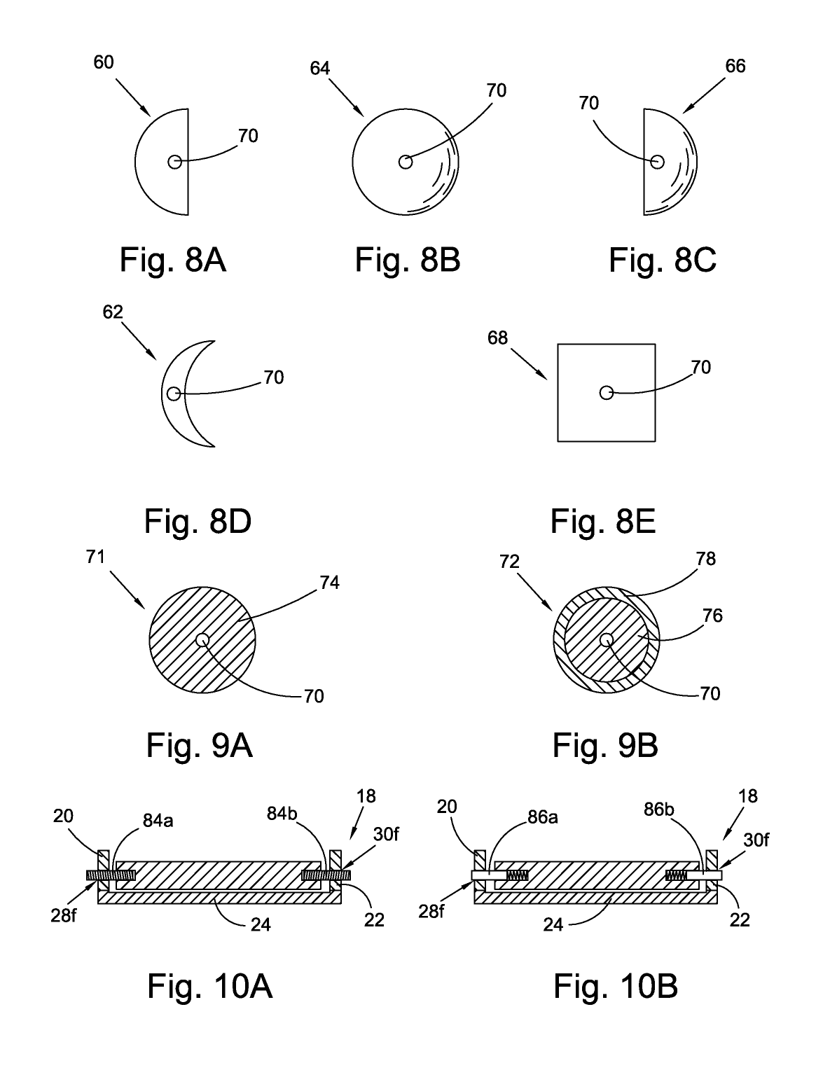

FIGS. 8A through 8E each show a side elevational view of different embodiments of a support member for use in a present invention apparatus for maintaining an angular position of a leg. The following discussion is best understood in view of FIG. 1 through 8E. Support member 16 may be constructed in a variety of shapes depending on the user's preferences and needs. For example, support member 16 may be selected from the group consisting of: cylinder 58, hemi-cylinder 60, crescent cylinder 62, sphere 64, hemisphere 66, cuboid 68 and combinations thereof. As each support member must be secured or releasably secured to rigid frame 18, each of the embodiments of support member 16 includes through hole 70. Alternatively, as discussed infra, support member 16 may include other means to secure or releasably secure to rigid frame 18.

FIG. 9A shows a cross sectional view of an embodiment of a support member, i.e., support member 71, for use in a present invention apparatus for maintaining an angular position of a leg, while FIG. 9B shows a cross sectional view of another embodiment of support member, i.e., support member 72, for use in a present invention apparatus for maintaining an angular position of a leg. Support member 71 is but one embodiment of a support member that may be used in combination with the present invention apparatus. Support member 71 comprises a single material, i.e., material 74. For example, material 74 may be constructed from wood or a rigid plastic. Alternatively, support member 72 may also be used in combination with the present invention apparatus. Support member 72 comprises a core, i.e., core 76, enclosed within an outer shell, i.e., outer shell 78. It should be appreciated that core 76 and/or outer shell 78 may be constructed from a rigid or substantially rigid material. For example, core 76 may be constructed from wood or a rigid plastic material, while outer shell 78 may be constructed from cork, plastic or an elastomeric material.

FIG. 10A is a cross sectional view of apparatus 10 taken generally along Line 10A/B-10A/B of FIG. 5 having an embodiment of a means for securing support member 16 within rigid frame 18, while FIG. 10B is a cross sectional view of apparatus 10 taken generally along Line 10A/B-10A/B of FIG. 5 having another embodiment of a means for securing support member 16 within rigid frame 18. It should be appreciated that support member 16 may be releasably secured within rigid frame 18 via any one of a plurality of securing means. For example, support member 16 may be releasably secured within frame 18 via longitudinal shaft 80 disposed through support member 16 within through hole 70. Similarly, support member 16 may be releasably secured within frame 18 via integral threaded shafts 84a and 84b disposed at each end of support member 16. In like fashion, support member 16 may be releasably secured within frame 18 via spring loaded ball/pin detents 86a and 86b disposed at each end of support member 16. It should be appreciated that although pins 86a and 86b are shown in FIG. 10B, the pins may also be replaced by spherical structures, i.e., balls.

First and second securing straps 38 and 40, respectively, may be constructed from a variety of materials. For example, in some embodiments, securing straps 38 and 40 are constructed from material comprising portions of hook and loop type fasteners. In these embodiments, securing straps 38 and 40 are inserted through buckles 88 and 90, respectively, drawn tight and subsequently hook portions 92 are affixed to loop portions 94. In like fashion, in other embodiments, each of securing straps 38 and 40 comprises a plurality of holes disposed along at least a portion of each strap and retaining means is disposed at a terminal end of each strap. It should be appreciated that the foregoing example of a securing strap functions and takes an analogous form as a belt and belt buckle. Thus, the portion of the strap comprising the plurality of holes is inserted within the retaining means, e.g., a retaining buckle, and tightened to a sufficient level and a portion of the retaining means passes through one of the plurality of holes thereby retaining the strap in a fixed position.

It should be appreciated that the present invention apparatus may also include additional means for maintaining the angular position of leg 12. Securing strap 38 may comprise holes 96a, 96b and 96c, while securing strap 40 may comprise holes 98a, 98b and 98c. By altering which of holes 96a, 96b and 96c and which of holes 98a, 98b and 98c attach securing straps 38 and 40, respectively, to frame 18, the length of each of securing straps 38 and 40 available for positioning behind leg 12 may be modified. By modifying these lengths, the position of the upper and lower portions of leg 12 relative to support member 36 may be changed. Thus, as additional length is provided, leg 12 may be bent further, i.e., a smaller angular position, while as length is removed, leg 12 may be bent less, i.e., a larger angular position. In view of the foregoing, it should be apparent to one having ordinary skill in the art that the present invention apparatus may maintain the angular position of a leg using a variety of means. Furthermore, although nuts and bolts are shown in the figures for securing the various elements to frame 18, one of ordinary skill in the art will appreciate that other attachment means are also possible, e.g., rivets or pins with locking elements, and such variations are within the spirit and scope of the claimed invention.

It should be further appreciated that the present invention also broadly comprises a method of maintaining an angular position of a leg having a knee. The method comprises the following steps. First, a rigid frame comprising oppositely disposed first and second side walls and an integral connecting member therebetween is provided. The integral connecting member is arranged to connect at least a portion of the first side wall to at least a portion of the second side wall and to be positioned behind the knee. Next, a plurality of through holes in each of the first and second side walls is provided. The plurality of through holes of the first sidewall are in registered alignment with the plurality of through holes of the second side wall. Then, a substantially rigid support member is provided and releasably secured to one of the plurality of through holes of the first side wall and one of the plurality of through holes of the second side wall, and the support member is positioned behind the knee. Next, first and second securing straps are provided. The first securing strap is releasably securable to the upper portions of the first and second side walls, while the second securing strap is releasably securable to the lower portions of the first and second side walls. The first strap is adapted to be releasably secured about a portion of the leg above the knee, while the second strap is adapted to be releasably secured about a portion of the leg below the knee. Then, the connecting member and the substantially rigid support member are positioned behind the knee. Lastly, the first and second securing straps are affixed about the leg. The foregoing method permits modifying the position of the substantially rigid support member relative to the first and second side walls, which in turn modifies the angular position of the leg.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

What I claim is:

1. An apparatus for maintaining an angular position of a leg having a knee, said apparatus comprising:
a rigid frame comprising oppositely disposed first and second side walls and an integral connecting member therebetween, said integral connecting member arranged to connect at least a portion of said first side wall to at least a portion of said second side wall and to be positioned behind said knee;
each of said first and second side walls comprises a plurality of through holes, said plurality of through holes of said first sidewall in registered alignment with said plurality of through holes of said second side wall;
a substantially rigid support member releasably secured to one of said plurality of through holes of said first side wall and one of said plurality of through holes of said second side wall, said support member positioned behind said knee; and,
first and second securing straps, said first securing strap releasably secured to upper portions of said first and second side walls and said second securing strap releasably secured to lower portions of said first and second side walls, said first strap adapted to be releasably secured about a portion of said leg above said knee and said second strap adapted to be releasably secured about a portion of said leg below said knee, wherein modifying a position of said substantially rigid support member relative to said first and second side walls modifies said angular position of said leg.

2. The apparatus of claim 1 wherein said first and second side walls and said connecting member form a substantially U-shaped cross section.

3. The apparatus of claim 1 wherein a shape of said substantially rigid support member is selected from the group consisting of: a cylinder, a hemi-cylinder, a crescent cylinder, a sphere, a hemisphere, a cuboid and combinations thereof.

4. The apparatus of claim 1 wherein each of said first and second side walls is arranged on opposite sides of said leg above and below said knee.

5. The apparatus of claim 1 wherein each of said plurality of through holes in said first side wall is arranged at a unique horizontal and/or vertical position relative to the others of said plurality of through holes in said first side wall and each of said plurality of through holes in said second side wall is arranged at a unique horizontal and/or vertical position relative to the others of said plurality of through holes in said second side wall.

6. The apparatus of claim 5 wherein said unique positions of said plurality of through holes permits modifying the position of said substantially rigid support member.

7. The apparatus of claim 1 wherein said substantially rigid support member is releasably secured to said first and second side walls via a securing means, said securing means selected from the group consisting of: a longitudinal shaft disposed through said support member, an integral threaded shaft disposed at each end of said support member, a spring loaded ball detent disposed at each end of said support member, a locking pin disposed in each end of said support member and combinations thereof.

8. The apparatus of claim 1 wherein said substantially rigid support member comprises a single material.

9. The apparatus of claim 1 wherein said substantially rigid support member comprises a core enclosed within an outer shell.

10. The apparatus of claim 1 wherein each of said first and second securing straps comprises hook and loop type fasteners.

11. The apparatus of claim 1 wherein each of said first and second securing straps comprises a plurality of holes disposed along at least a portion of said strap and a retaining means disposed at a terminal end of said strap.

12. A method of maintaining an angular position of a leg having a knee, said method comprising the steps of:
a) providing a rigid frame comprising oppositely disposed first and second side walls and an integral connecting member therebetween, said integral connecting member arranged to connect at least a portion of said first side wall to at least a portion of said second side wall and to be positioned behind said knee;
b) providing a plurality of through holes in each of said first and second side walls, said plurality of through holes of said first sidewall in registered alignment with said plurality of through holes of said second side wall;
c) providing a substantially rigid support member releasably secured to one of said plurality of through holes of said first side wall and one of said plurality of through holes of said second side wall, said support member positioned behind said knee;
d) providing first and second securing straps, said first securing strap releasably securable to upper portions of said first and second side walls and said second securing strap releasably securable to lower portions of said first and second side walls, said first strap adapted to be releasably secured about a portion of said leg above said knee and said second strap adapted to be releasably secured about a portion of said leg below said knee;
e) positioning said connecting member and said substantially rigid support member behind said knee; and,
f) affixing said first and second securing straps about said leg, wherein modifying a position of said substantially rigid support member relative to said first and second side walls modifies said angular position of said leg.
